(19) United States
(12) Patent Application Publication
(10) Pub. No.: US 2019/0099550 A1
(43) Pub. Date: Apr. 4, 2019

(54) DEVICES FOR STABILIZING MEDICAL TUBES

(71) Applicant: IVY DEVICES INC., Grande Prairie (CA)

(72) Inventors: Frederick Robin ESTLIN, County of Grande Prairie (CA); William K. REILLY, Cochrane (CA)

(21) Appl. No.: 15/724,823

(22) Filed: Oct. 4, 2017

(51) Int. Cl. A61M 5/14; F16L 3/223

(52) U.S. Cl. CPC A61M 5/1418; A61G 7/0503; F16L 3/2235

(57) ABSTRACT

A medical line stabilizer is provided and includes an elongate rigidifying body having an axis, and multiple open channels that extend parallel to the axis and are adapted to receive a medical line, such as intravenous tubing. The open channels have notches configured to allow controlled bending of the body, which helps to prevent medical lines from becoming entangled or bent.

### BACKGROUND

The present invention relates to a medical line stabilizer that serves to stabilize various types of medical lines and, in particular, intravenous tubing.

It is common for hospitalized patients to be fitted with medical lines. In some cases, patients receive medication or blood products through intravenous tubing (commonly referred to as an “IV”). In other cases, patients may require N/G, O/G, broviac, oxygen, external pacing or monitor wiring, or some other type or form of medical line. Without constant monitoring from a health care provider, these lines can kink; thereby disrupting the flow of blood product, medication or oxygen to the patient. In some cases, these lines can entangle the patient creating a hazard. There have been documented cases of patients being strangled after becoming entangled in medical lines.

US 2015/0276091 (Jaeker) discloses a line guide device for protectedly guiding at least one line, in particular a capillary tube, is produced in longitudinal portions or completely in one piece and preferably from plastic.

U.S. Pat. No. 5,316,246 (Scott et al.) discloses an intravenous tube holder having positioned along one edge, a plurality of clips for securing the holder to an intravenous tube.

U.S. Pat. No. 5,703,330 (Kujawski) discloses a carrier for a wire harness and fuel brake, or other tubes of a vehicle with inner and outer channels each having a generally C-shape cross section and constructed to releasably snap together to form a duct in which the wire harness is received. Snap clips on the exterior of the channels releasably retain the tubes.

U.S. Pat. No. 7,198,066 (Kagenow) discloses relates to an anti-kinking device, in particular for supporting and stabilizing an infusion tubing for fluid infusion in a safety loop, comprising means for receiving and fixing the infusion tubing in connection with a catheter.

US 2015/0034776 (St. John et al.) discloses a line management device includes a body with a slot for receiving a tube and a base adapted to mount the body to a support surface.

US 2014/0031788 (Sung et al.) discloses exoskeletal devices or sleeves that can be used with the delivery tube of an applicator device to help dispense fluids. The exoskeletal devices or sleeves are rigid, pre-shaped, and snappably or slidably affixed to the delivery tube. The exoskeletal devices or sleeves do not come into contact with the flowable biocompatible fluid being dispensed, and they can be placed at any position along the delivery tube to address different application situations.

US 2004/199142 (Reilly) discloses a medical line stabilizer includes an elongate rigidifying body having an axis. An open channel extends parallel to the axis which is adapted to receive a medical line, such as intravenous tubing. The medical line stabilizer is secured to (e.g. around) the medical line with tape to prevent the medical line from kinking or tangling.

JP2012040145 (Koichi) discloses a protective cover which includes slits installed in an axial direction and a cylindrical cover member in which a medical tube can be stored inside through the slits.

U.S. Pat. No. 5,427,338 (Garrett) discloses a device includes an elongated, rigid base plate having upstanding wall sections; the wall sections include axially spaced apart, differently configured openings for receiving different patient care lines.

### SUMMARY

In an embodiment, a medical line stabilizer is provided and includes a rigidifying elongate body having a body axis, multiple open channels extending parallel to the body axis, where each channel has a base and opposing walls adapted to receive a medical line extending along the body axis, and where the walls include notches configured to allow controlled bending of the body about a bending axis which is transverse to the body axis and to the wall notches.

The medical line stabilizer may comprise pairs of wall notches to allow controlled bending of the channel base at a particular axial position.

The medical line stabilizer may comprise one or more quartets of mutually opposing wall notches, each quartet being configured to allow controlled bending of the channel base at a particular axial position.

The notches may comprise opposed angled sides, wherein bending is restricted when the opposed angled sides impinge on one another.

The medical line stabilizer may comprise two opposed open channels. That is, the cross section of the medical line stabilizer may form an H shape.

The notches may extend substantially to the base of the channel.

The medical line stabilizer may comprise two open channels in a side-by-side configuration. That is, the cross section of the medical line stabilizer may form an E shape.

The medical line stabilizer may comprise notches in each of the walls notches to allow bending of the channel base at a particular axial position.

The length of each notch along the body axis may be less than twice the notch depth.

The medical line stabilizer may comprise wall grooves configured to allow torsion of the body about the body axis.

The length of each groove along the body axis may be more than four times the groove depth.

According to another embodiment, a medical line stabilizer is provided for stabilizing medical tubes and includes a rigidifying elongate body and two or more clips attached to, and arranged axially along, the elongate body, where the clips form multiple medical line channels configured such that multiple medical lines can be anchored along axes parallel to the elongate body by each medical line being received by a respective medical line channel associated with at least two of the clips.

The elongate body may comprise a medical-line channel.

The medical-line channel may be configured such that a medical line can be removed from the elongate body independently of the clips.

The clips may be permanently affixed, or integrally connected, to the elongate body.

The clips may be releasably affixed to the elongate body.

The stabilizer may be made of food-grade plastic.

The body may be transparent. This may help facilitating visual inspection of the medical line positioned in the channel.

The medical line stabilizer may form an integral part of a medical (e.g. IV) line. For example, the stabilizer and the line may be of unitary construction. The advantages of an integral line may include that a practitioner may spend less time in installation and removal. It may also reduce the possibility of the line becoming detached. This structure may be more efficient in manufacturing and so offer potential cost reductions.

Viewing windows or grooves may be positioned along the length of the body to facilitate visual inspection of intravenous tubing positioned in the channel. Alternatively, the stabilizer body may be transparent so that fluids may be seen through the body of the medical line stabilizer.

The body may be made from food grade polymer plastic that will not harm the patient if portions of the body should be ingested.

A medical line stabilizer may comprise 1-5 bending points (or 3-5), each bending point controlled by one or more notches. A medical line stabilizer may comprise 1-5 torsion points (or 3-5), each torsion point controlled by one or more grooves.

The body may be color coded to give a visual indication of the type of fluid passing through the intravenous tubing.

The body may comprise integral closures positioned at spaced intervals. This may help prevent accidental removal of intravenous tubing from the channel.

Although beneficial results may be obtained through the use of the medical line stabilizer, as described above, often lengths of tubing are connected in end to end relation by connectors. An axial force exerted upon the tubing, however, can result in lengths of tubing separating at the connectors. Should the connectors separate there is a danger of an air embolism, cross-contamination, or a back flow of body fluid. An example of such an air embolism is the death of Andrina Hansen following an IV-related incident at Mt. Sinai Hospital in 1991. An IV line disconnected at a connection fitting between IV “end to end” connectors, resulting in an air embolism entering into Andrina's catheter and traveled to her brain, causing brain damage. The channel may have at least one connector receiving cavity adapted to receive an intravenous tubing connector. The cavity may have opposed contact shoulders which limit axial movement of the connector. In the event an axial force is exerted upon the intravenous tubing, the force is transmitted via one of the shoulders to the body of the medical line stabilizer which prevents the connector from separating.

For the purposes of this disclosure, a notch may be considered an indentation or incision from the top of a wall down. For the purposes of this disclosure, a groove may be considered an elongate indentation in a wall which extends along the body axis of the medical line stabilizer. The elongations may serve as a taping points for securing the medical lines into the stabilizer.

### BRIEF DESCRIPTION OF THE DRAWINGS

Various objects, features and advantages of the invention will be apparent from the following description of particular embodiments of the invention, as illustrated in the accompanying drawings. The drawings are not necessarily to scale, emphasis instead being placed upon illustrating the principles of various embodiments of the invention. Similar reference numerals indicate similar components.

FIG. 1a is a side perspective view of a first embodiment of a medical line stabilizer in an unbent configuration.

FIG. 1b is a side perspective view of the first embodiment of the medical line stabilizer of FIG. 1a in a bent configuration.

FIG. 2a is a side view of a second embodiment of a medical line stabilizer in an unbent configuration.

FIG. 2b is a perspective view of the second embodiment of the medical line stabilizer of FIG. 2a.

FIG. 2c is a transverse cross-section view of the second embodiment of the medical line stabilizer taken substantially along the line 2c-2c and in the direction generally indicated in FIG. 2a.

FIG. 2d is a fragmentary, side view of the second embodiment of the medical line stabilizer of FIG. 2a with exemplary dimensions.

FIG. 2e is a side view of a portion of the second embodiment of the medical line stabilizer of FIG. 2a with exemplary dimensions.

FIG. 2f is a transverse cross-section view of the second embodiment of the medical line stabilizer taken substantially along the line 2c-2c and in the direction generally indicated in FIG. 2 with exemplary dimensions.

FIG. 3a is a perspective view of a third embodiment of a medical line stabilizer.

FIG. 3b is a transverse cross-section view of the third embodiment of the medical line stabilizer taken substantially along the line 3b-3b and in the direction generally indicated in FIG. 3a.

### DETAILED DESCRIPTION

### Introduction

Health care professionals need to ensure that the medical line is free of kinks and that fluid is flowing properly through the intravenous tubing. In addition, checks may be performed periodically to ensure that there are no air bubbles in the medical line. Therefore, the inventors have identified a need for a partially flexible medical line stabilizer which controls the extent to which a medical line can be bent.

Various aspects of the invention will now be described with reference to the figures. For the purposes of illustration, components depicted in the figures are not necessarily drawn to scale. Instead, emphasis is placed on highlighting the various contributions of the components to the functionality of various aspects of the invention. A number of possible alternative features are introduced during the course of this description. It is to be understood that, according to the knowledge and judgment of persons skilled in the art, such alternative features may be substituted in various combinations to arrive at different embodiments of the present invention.

### First Embodiment

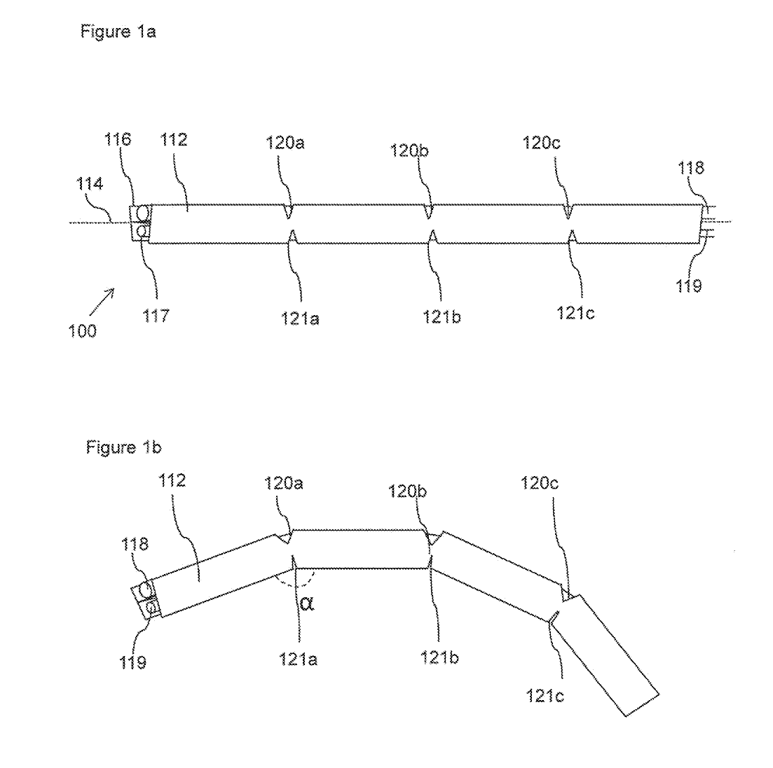

FIGS. 1a and 1b show a first embodiment of a medical line stabilizer. Referring to FIG. 1a, there is provided a medical line stabilizer 110, which includes an elongate rigidifying body 112 of food grade polymer plastic (e.g. Elastollan® 1154D) with an axis 114. Two open channels 116, 117 extend parallel to axis 114. Each open channel 116, 117 is adapted to receive a medical line, such as intravenous tubing 118, 119. Intravenous tubing 118 is secured in place within channel 116 and intravenous tubing 119 is secured in place within channel 117.

In this case, body 112 of medical line stabilizer 10 is transparent, thereby facilitating visual inspection of intravenous tubing 18 that is positioned in channel 16, however it will be appreciated that body 12 could also be color coded to give a visual indication of the type of fluid passing through intravenous tubing 18. The medical line stabilizer may be formed from food grade polymer plastic.

In this embodiment, each open channel has a base and opposing walls. The open channel allows the medical line to be easily inserted and removed from the body of the medical line stabilizer. In this embodiment, the walls comprise notches 120a-c, 121a-c configured to allow controlled bending of the body about a bending axis transverse (e.g. perpendicular) to the body axis and to the notches (the bending axis may be in the plane of the channel base). That is, the bending is facilitated by the notches opening and closing, the notches being configured to be transverse to the body axis. As shown in FIG. 1a, when the body axis and notches are aligned with the page, the bending axis is transverse (perpendicular or normal) to the page.

The two channels are opposed to each other in this embodiment in an H configuration (i.e. the transverse cross-section of the medical-line stabilizer is shaped like the letter H). That is, the two channels share a common base section and the walls extend from this common base in opposite directions.

### Operation

The use and operation of medical line stabilizer 100 will now be described with reference to FIGS. 1a and 1b. Referring to FIG. 1a, in this case, during hospitalization, when a patient is fitted with intravenous tubing 118, 119, the intravenous tubing 118, 119 is secured to medical line stabilizer 100 by being inserted between the walls of an open channel 116, 117 and optionally by tape. The medical line stabilizer 100 serves to help prevent intravenous tubing 118, 119 from kinking or tangling by stiffening the medical line assembly. Although medical line stabilizer 100 is sufficiently rigid so as to prevent kinking or tangling of IV tubing 118, 119 medical line stabilizer 100 has some moderate flexibility allowing it to bow or bend slightly, such that a patient will not be injured if they roll onto it or inadvertently poke themselves with it. The medical line may be resilient or elastic in addition to being moderately flexible. Alternatively or in addition, the medical line stabilizer may be configured to allow plastic deformation (e.g. the material used may be a plastically deformable material). This may allow the stabilizer to be bent into a particular shape (e.g. to hang over a bed rail).

As shown in FIG. 1b, the notches control the bending of the medical line by providing particular points where bending is facilitated and by restricting the extent of bending at these points (or axial positions). The spacing between successive notches (or bending points) may be more than 5 times the length of the notch (greater or equal to 10 times the length of the notch). That is, the notches may be spaced apart such that there is a region between the notches which are not affected by the bending at the axial position of the notch. These inter-notch sections may be configured to remain substantially straight whilst the stabilizer is being bent. This may help control the degree of bending at particular points along the medical line stabilizer body axis. The length of the notch may be considered to be the maximum length of the notch at which the two notch sides can impinge on the each other when bent measured when the body axis is in a straight configuration. For notches with straight angled sides, this will be the length at the top of the notch (furthest from the base).

In this case, the medical line stabilizer channels comprise pairs of wall notches to allow controlled bending of the channel base at a particular axial position. In particular, bending is facilitated at particular axial positions by arranging the notches on the two opposing channels in quartets. That is, the notches in each quartet are positioned close enough to each other so that when the medical line stabilizer is bent, the notches work in consort to facilitate bending. That is, when the medical line stabilizer is bent about a bending axis transverse to the walls (e.g. perpendicular to the body axis and parallel to the plane of the base), the notch pair of one channel opens, and the notch pair of the opposing channel closes.

The closing notch pair restricts or limits the extent of bending when the sides of the notches impinge on one another. That is, before the sides of the notch impinge, bending is facilitated substantially by bending of the channel base. After the sides of the notch impinge, further bending would require deformation of the walls themselves. This increase in bending force due to the different mode of deformation restricts the extent of bending.

By having opposed channels with corresponding notches on each channel, the extent of bending can be restricted for bending in either direction about the wall axis. In this way, each notch quartet can be configured to allow controlled bending of the channel base at a particular axial position.

The notches may be configured to allow bending up to an internal angle, a, of 120° (or 90°). The notches may be configured such that the diameters of curvatures of the medical lines 118, 119 do not fall below 5¼ inches (13 cm).

It will be appreciated that, in this case, the notches extend substantially to the base of the channel. This means that the bending of the medical line stabilizer is facilitated by bending the base rather than significantly stretching the material of the walls.

The length of each notch (or slit) along the body axis in this case is less than twice the notch depth. The notch angle (the angle between the two notch sides) in this case is 30°. Other embodiments may have notch angles of between 20° and 45°.

Once intravenous tubing 118, 119 is secured in channel 116, 117 of body 112, nursing staff are able to view intravenous tubing 118, 119 by looking at open channel 116 or though transparent body 112 of medical line stabilizer 100 to monitor the flow of fluids through intravenous tubing 118, 119. When medical line stabilizer 100 is being used with infants, it is preferred that body 112 be made from food grade polymer plastic so that it will not harm a patient if portion of body 112 is inadvertently ingested. This can be of particular concern where the patient is an infant as infants have a tendency to put things in their mouths.

Lengths of tubing may be connected in end to end relation by connectors. To prevent separation, the medical line stabilizer may comprise opposed contact shoulders in notches or grooves which limit axial movement of connectors. If a force is exerted upon intravenous tubing, it will tend to slide along channel. The movement of intravenous tubing may be halted when one of connectors engages one of shoulders. When further axial force is exerted on intravenous tubing, the force is transmitted via one of shoulders to body of medical line stabilizer which prevents connectors from separating.

### Second Embodiment

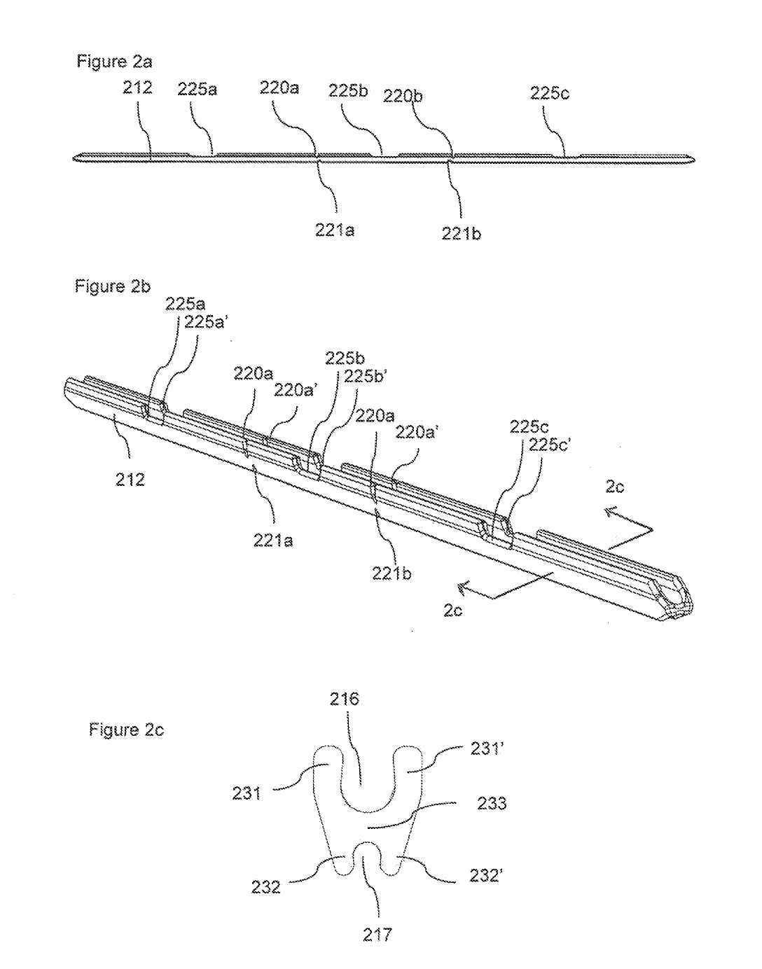

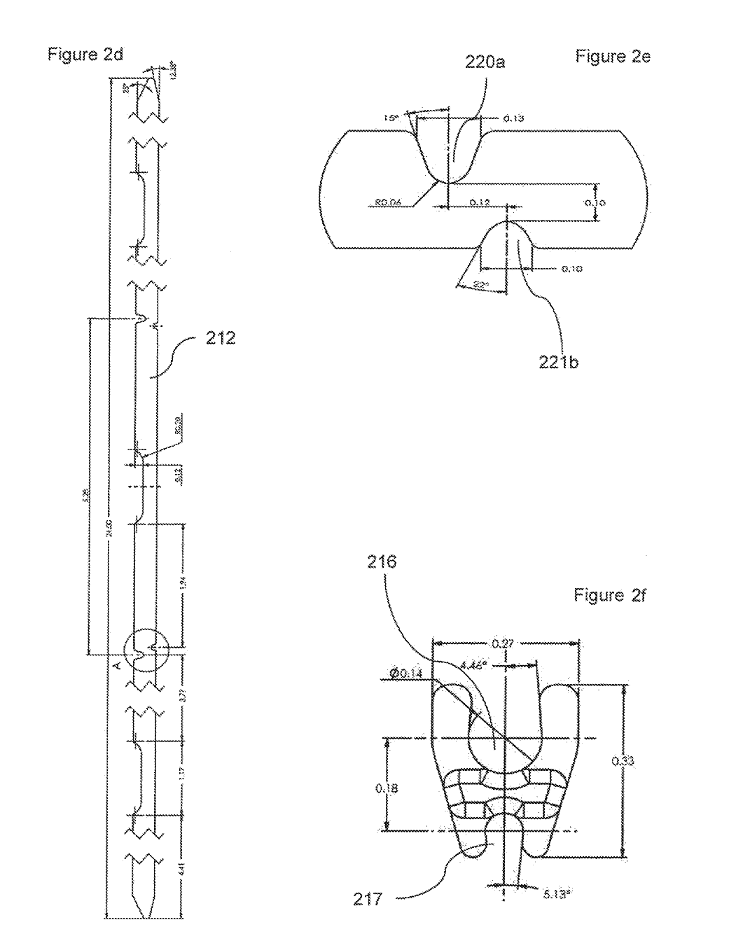

The use and operation of a second embodiment of medical line stabilizer 200 will now be described with reference to FIGS. 2a-2f. As with the embodiment of FIG. 1a, during hospitalization, when a patient is fitted with intravenous tubing 218, medical line stabilizer is secured to intravenous tubing using open channels.

In this case, in addition to notches, this embodiment also includes grooves positioned in the walls. The grooves form viewing windows which allow inspection of fluid in the medical lines contained within the channels. In this case, the grooves extend along a greater axial distance along the body than the notches.

As shown in FIGS. 2a-2c, the medical line stabilizer includes a rigidifying elongate body 212 having a body axis and multiple open channels 216-217 extending parallel to the body axis, each of which is adapted to receive a medical line (not shown) extending along the body axis, where each channel has a base 233 and opposing walls 231, 231′, 232, 232′, and where the walls comprise notches 220a, 220a′, 220b, 220b′, 221a, 221a′, 221b, 221b′ configured to allow controlled bending of the body about a bending axis transverse the body axis in the plane of the base.

The base 233 and walls 231, 231′ of first channel 216 are shaped to accommodate a standard size IV line with a diameter of 0.14 inches. The walls of the first channel 231, 231′ slope slightly in (4.5° from vertical) to ensure that the medical line is held securely in place. The base 233 and walls 232, 232′ of second channel 217 are shaped to accommodate a micro size IV line. The walls of the second channel 232, 232′ slope slightly in (5.1° from vertical) to ensure that the medical line is held securely in place. The height of the medical line stabilizer is ⅓ of an inch (other embodiments may have a height of between ¼ and 1 inch). The length of the medical line stabilizer in this case is 24 inches (other embodiments may be between 12 and 36 inches in length). The separation between successive notches in this case is around 5 inches (in other embodiments the separation distance may be between 3 and 10 inches).

As in the embodiment of FIG. 1a, the medical line stabilizer of this embodiment comprises two quartets of mutually opposing (e.g. exactly opposing or slightly offset) wall notches, each quartet being configured to allow controlled bending of the channel base at a particular axial position.

The configuration of the notches can be seen in more detail in FIG. 2e. The notches 220a in the standard line channel are shaped with a round bottom (of radius 0.06 inches) which merges into sides which angle away from a vertical at an angle of 15°. The length of the notch at the top along the body axis is 0.13 inches. The notches 220a in the micro line channel are shaped with a round bottom (of radius 0.06 inches) which merges into sides which angle away from a vertical at an angle of 22°. The length of the notch at the top along the body axis is 0.1 inches. Within the quartet, the notch pair on one channel is axially offset from the notch pair on the other channel by 0.12 inches. The bottoms of the notch pairs for each channel are vertically offset by 0.1 inches (vertically in this case is defined with respect to a horizontal plane which is parallel to the bending axis and the body axis).

Offsetting the notches on the line stabilizer may help strengthen the integrity of the stabilizer itself. The inventors have found that slightly misaligning the notches in the mold causes the notches to be offset which aided in the strength and integrity of line stabilizer. The degree of offset may also allow the bending resistance to be controlled as the inventors found that having the notches exactly aligned makes the stabilizer easier to bend. The offset may be between 0.05-0.2 inches.

As in the embodiment of FIG. 1a, bending is restricted when the opposed angled sides impinge on one another.

In this case, the medical line stabilizer comprises wall grooves 225a, 225a′, 225b, 225b′, 225c, 225c′ configured to allow torsion of the body about the body axis. The length of each groove along the body axis, in this embodiment, is more than four times the groove depth. These grooves may act as windows to allow users (e.g. nurses or doctors) to monitor flow through the medical lines. The grooves may be configured to allow the medical lines within the channels to be taped in place using tape.

There medical device may be configured such that there are no sharp edges on body 112 that could potentially cause abrasions or cuts to the patient. All edges should be made smooth.

### Modular Medical Line Stabilizer

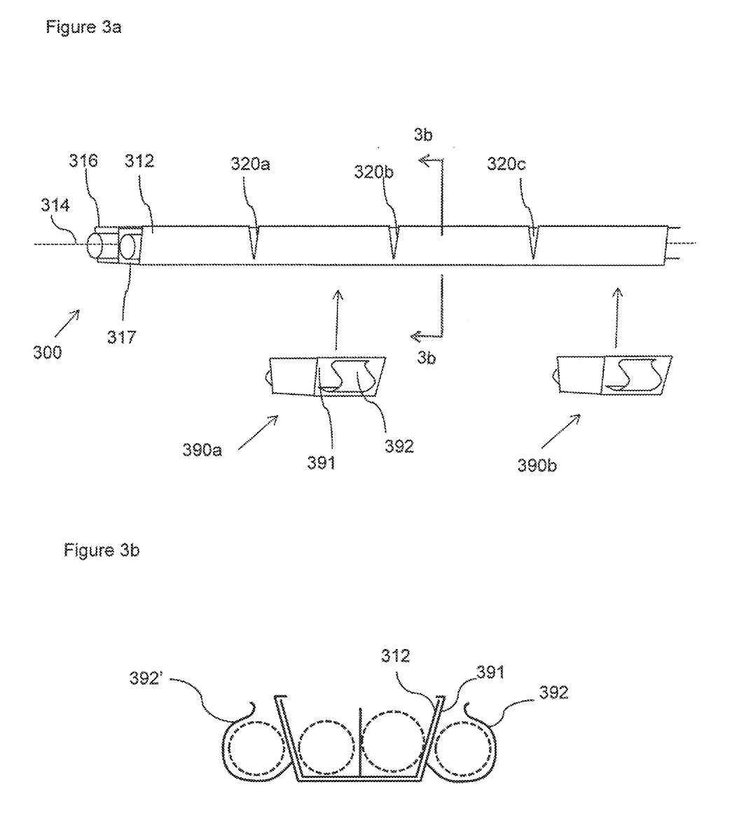

As shown in FIGS. 3a-3b, in some embodiments, a medical line stabilizer includes two or more removable clips 390a, 390b attached to, and arranged axially along, the elongate body, where the clips form one or more additional medical line channels configured such that one or more medical lines can be anchored along axes parallel to the elongate body axis 314 by each medical line being received by a respective medical line channel associated with at least two of the clips.

An example of this type of medical line stabilizer is shown in FIGS. 3a-3b. In this embodiment 300, the medical line stabilizer comprises two open channels 316, 317 in a side-by-side E configuration (i.e., the transverse cross-section of the medical-line stabilizer is shaped like the letter E). That is, the two channels share a common wall and the base sections extend from this common wall in opposite directions. Notches 320a-c are provided in the channel walls to facilitate controlled bending.

The clips each comprise a connector 391 configured to connect to the body 312 of the medical line stabilizer 300; and one or more medical line holders 392, 392′. When the clip 390a is connected to the body 312 of the medical line stabilizer 300 (see FIG. 3b), the axis of the one or more medical line holders 392, 392′ lie parallel to the body axis 314 of the medical line stabilizer. This means that more medical lines can be arranged in parallel than would be the case for just the medical line stabilizer without the additional clips. Multiple clips may be required to support the additional medical lines along their length.

In clip embodiments, the central body may not have a channel but may be, for example, simply a solid rod.

The clips may be permanently affixed, or integrally connected, to the central body.

The ends of the channels may be tapered and smoothed to prevent the lines being pinched.

The medical line clips may be color-coded to enable identification of different medical lines (e.g. the feed tube may be in the red channels and an oxygen tube may be in the blue channel).

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

Although the present invention has been described and illustrated with respect to preferred embodiments and preferred uses thereof, it is not to be so limited since modifications and changes can be made therein which are within the full, intended scope of the invention as understood by those skilled in the art.

1. A medical line stabilizer comprising:
a rigidifying elongate body having a body axis;
multiple open channels extending parallel to the body axis, each channel having a base, opposing walls and an elongate opening extending along the body axis and being adapted to receive a medical line extending along the body axis, wherein the walls comprise notches configured to allow controlled bending of the body about a bending axis which is transverse to the body axis and to the notches, and wherein each of said channels is configured to allow each received medical line to be independently removed through the respective elongate opening.

2. The medical line stabilizer according to claim 1, wherein the medical line stabilizer comprises pairs of wall notches to allow controlled bending of the channel base at a particular axial position.

3. The medical line stabilizer according to claim 1, wherein the medical line stabilizer comprises one or more quartets of mutually opposing wall notches, each quartet being configured to allow controlled bending of the channel base at a particular axial position.

4. The medical line stabilizer according to claim 1, wherein the notches comprise opposed angled sides, wherein bending is restricted when the opposed angled sides impinge on one another.

5. The medical line stabilizer according to claim 1, wherein the medical line stabilizer comprises two opposed open channels.

6. The medical line stabilizer according to claim 1, wherein the notches extend substantially to the base of the channel.

7. The medical line stabilizer according to claim 1, wherein the medical line stabilizer comprises two open channels in a side-by-side configuration.

8. The medical line stabilizer according to claim 1, wherein the medical line stabilizer comprises notches in each of the walls notches to allow bending of the channel base at a particular axial position.

9. The medical line stabilizer according to claim 1, wherein each of said notches has a length and a depth, and wherein the length of each of said notches along the body axis is less than twice the depth of each of said notches.

10. The medical line stabilizer according to claim 1, wherein the medical line stabilizer comprises wall grooves configured to allow torsion of the body about the body axis.

11. The medical line stabilizer according to claim 10, wherein said wall grooves are configured to allow the medical lines within the channels to be taped in place.

12. A medical line stabilizer for stabilizing medical tubes comprising:
a rigidifying elongate body with a longitudinal body axis along said elongate body, said elongate body configured to receive at least one medical line; and
two or more clips attached to the elongate body, the two or more clips being spaced apart along the longitudinal body axis of the elongate body,
wherein the clips form multiple medical line channels configured such that multiple medical lines can be anchored along their length along axes parallel to, and outside of, the elongate body by each medical line being received by a respective one of said medical line channels associated with at least two of the clips.

13. The medical line stabilizer of claim 12, wherein the elongate body comprises a medical-line channel.

14. The medical line stabilizer according to claim 13, wherein the medical-line channel is configured such that a medical line can be removed from the elongate body independently of the clips.

15. The medical line stabilizer according to claim 12, wherein the clips are permanently affixed, or integrally connected, to the elongate body.

16. The medical line stabilizer according to claim 12, wherein the clips are releasably affixed to the elongate body.

17. The medical line stabilizer according to claim 1, wherein the stabilizer is made of food-grade plastic.

18. The medical line stabilizer according to claim 1, wherein the body is transparent, thereby facilitating visual inspection of the medical line positioned in the channel.
